(19) United States
(12) Patent Application Publication
(10) Pub. No.: US 2005/0244438 A1
(43) Pub. Date: Nov. 3, 2005

(54) Simple and multiple emulsions for decontamination of an organism or surfaces

(76) Inventors: Elise Mona-Lydia Dobin-Assouly, Antony (FR); Jean-Louis Grossiord, Saint-Germain-Les-Corbeil (FR); Dominique Nicole Michele Pareau, Verriere Le Buisson (FR); Monqiue Anne Seiller-Boutin, Sceaux (FR); Moncef Stambouli, Chatenay Malabry (FR)

Correspondence Address: YOUNG & THOMPSON, 745 SOUTH 23RD STREET, 2ND FLOOR, ARLINGTON, VA, 22202, US

(21) Appl. No.: 10/516,914

(22) PCT Filed: Jun. 4, 2003
(86) PCT No.: PCT/FR03/01674

§ 371 Date: May 31, 2005

(30) Foreign Application Priority Data
Jun. 4, 2002 (FR) 02/06849

(51) Int. Cl. A61K 9/00; C07C 69/00

(52) U.S. Cl. 424/400; 560/143

(57) ABSTRACT

The present invention relates to the use of simple or multiple emulsions comprising in their organic phase one or more extractant compounds capable, when said emulsions are brought into contact with an external medium, either biological such as gastric liquid, skin or blood, or artificial such as metal or plastic surfaces, of extracting from said external medium specific toxic molecules capable of binding to said extractant or extractants, for detoxication either by oral, topical or parenteral route, or by simple application to the abovementioned surface.

A subject of the invention is novel simple water-in-oil, or multiple water-in-oil-in-water emulsions, and their use for the detoxication of an organism or of surfaces.

Acute intoxications represent, at the beginning of this millennium, one of the prime causes of hospitalization in the developed countries and of death of individuals under the age of thirty years in developing countries.

Treatment of oral intoxications is often very difficult to implement, as it is generally difficult to obtain the history of the intoxication from the patient himself. The clinician must essentially pay attention to the symptoms in order to be able to identify the toxic compound ingested and thus establish the appropriate treatment. The latter will essentially depend on the nature of the toxic molecule(s), the subject, the time of ingestion, the seriousness of the intoxication and the clinical signs.

In the case of oral intoxications, the treatments implemented at the present time to avoid the passage of toxic substances into the blood involve either gastric lavage (which is proscribed in the case of corrosive products and which has risks with regard to respiration), or activated carbon (the effectiveness of which has not been indisputably demonstrated and which, for this reason is used for want of something better and in variable manner depending on the hospitals).

In the case of cutaneous intoxications, at the present time no universal medicament exists which is well tolerated and has a rapid action.

The phenomenon of extraction of toxic compounds by simple and multiple emulsions has already been known for several years. The use of simple oil/water emulsions for the purification of industrial effluents by extraction has already been implemented on numerous metallic or organic pollutants, using a suitable extractant/de-extractant pair. But the therapeutic use of these simple emulsions has never been envisaged. Moreover, these systems have a useful life which is intentionally very limited (necessary for rapid recovery and recycling of the constituents) which make them completely unsuitable for such an application. Moreover, the oral administration of the simple water/oil emulsions and multiple water/oil/water emulsions for the treatment of overdoses of medicaments was envisaged some twenty years ago, only at research level. But these emulsion systems contain no extractant, thus appreciably reducing the capture speed. Moreover, the multiple water/oil/water emulsions which have already been envisaged have a precarious stability.

In the face of this established fact of the relative ineffectiveness of the treatments currently implemented and given the evident implications in the field of public health, the invention makes it possible to provide an original and effective alternative in essential treatments intended for industrial, military, hospital, domestic and environmental emergencies.

Thus, the purpose of the invention is to allow the treatment of oral intoxications which originate from the ingestion of pharmaceutical and non-pharmaceutical products, but also cutaneous intoxications of domestic, industrial or military origin.

The purpose of the present invention is the use of emulsions which are very stable due to the presence of polymeric surfactants in these emulsions.

The present invention relates to the use of simple or multiple emulsions comprising in their organic phase one or more extractant compounds capable, when said emulsions are brought into contact with a medium, also designated as an external medium, either biological such as gastric liquid, skin or blood, or artificial such as metal or plastic surfaces, to extract from said medium specific toxic molecules capable of binding to said extractant or extractants, for the preparation of pharmaceutical compositions intended for the prevention or treatment of intoxications by oral, topical or parenteral route, or for the detoxication of surfaces by simple application to the abovementioned surfaces.

The present invention relates to the use as mentioned above, of simple water-in-oil, or multiple water-in-oil-in-water emulsions, the internal aqueous phase of which comprises one or more de-extractant compounds, trapping the toxic molecules extracted from the external medium.

By “de-extractant compound”, is meant a molecule which will react chemically with the complex formed by the toxic molecule and the extractant, which makes it possible on the one hand to regenerate the extractant, and on the other hand, to trap the toxic molecule.

The present invention also relates to the abovementioned use, characterized in that the extractant is chosen from:
amine derivatives, such as the primary, secondary or tertiary amines or quaternary ammonium salts, comprising one or more carbon chains each comprising approximately 1 to 18 carbon atoms, in particular trioctylamine or trilaurylamine, when the toxic molecule to be eliminated has an acid or anionic character,
organic acids, such as organophosphorated acids, thiophosphorated acids, carboxylic acids, comprising one or more carbon chains each comprising approximately 1 to 18 carbon atoms, when the toxic molecule to be eliminated has a basic or cationic character,
solvating molecules, such as alcohols, organophosphates, phosphine oxides, organosulphides or sulphoxides, comprising one or more carbon chains each comprising approximately 1 to 18 carbon atoms, when the toxic molecule to be eliminated has a neutral character.

The expression “molecule having an acid character” broadly designates a Lewis acid (electron acceptor) such as a weak acid, in particular acetic acid, lactic acid, citric acid, acetylsalicylic acid or hydrocyanic acid.

The expression “molecule having an anionic character” designates an aqueous anion such as for example the cyanide, fluoride or chloride ions, or anionic metal complexes such as for example FeCl₄⁻ or AuCl₄⁻.

The expression “molecule having a basic character” broadly designates a Lewis base (electron donor) possessing for example a nitrogen atom (protonable) such as oxine, urea, ammonia, quinine or the amphetamines, or possessing a sulphur atom (protonable) such as for example the sulphide ion or the sulphite ion.

The expression “molecule having a cationic character” designates an aqueous cation such as for example the ammonium ion or metal cations.

The expression “molecule having a neutral character” designates a molecule having no marked electron exchanger properties, such as for example the alcohols (ethanol), ketones, ethers, paracetamol etc.

A preferred organophosphorated acid, among the extractants, is di-2-ethylhexylphosphoric acid.

Among the alcohols, octanol or decanol are preferably used as extractants.

The present invention relates to the use as defined above, characterized in that the de-extractant is chosen from:
bases such as NaOH, KOH, Na₂CO₃, when the toxic molecule to be eliminated has an acid or anionic character,
ionic salts such as NaCl, NH₄Cl or NaNO₃, when the toxic molecule to be eliminated has an anionic character,
acids such as hydrochloric acid or lactic acid, when the toxic molecule to be eliminated has a basic or cationic character,
compounds which are oxide-reducing or chelating in character, such as chromium (VI) salts, thiourea, ethylene diamine tetracetic acid, chlorinated or fluorinated derivatives, ascorbic acid, when the toxic molecule to be eliminated has a neutral character.

The expression “compounds which are oxide-reducing or chelating in character” designates compounds capable of reducing or oxidizing the toxic substance or of forming with it a lipophobic complex.

According to an advantageous embodiment of the present invention, the invention relates to the use of simple water-in-oil emulsions comprising:
an external organic phase containing:
   one or more extractants, as defined above, the mass ratio of the extractant or extractants with respect to the organic phase being comprised between approximately 0.1 and approximately 20%,
   one or more lipophilic surfactants with an ether bond, such as the alkyl dimethicone copolyols, or with an amine bond such as long-chain condensed polyamines, or sorbitan or glycol esters, the mass ratio of the lipophilic surfactant or surfactants with respect to the organic phase being comprised between approximately 0.5 and approximately 20%,
   hydrocarbons (qs) such as liquid paraffins, perhydrosqualene or silicones or synthetic esters,
an internal aqueous phase containing one or more de-extractants as defined above, and optionally an additive such as an electrolyte or a sugar, the mass ratio of the internal aqueous phase with respect to the emulsion being comprised between approximately 1 and approximately 80%, preferably between approximately 20% and approximately 70%.

According to an advantageous embodiment of the present invention, the invention relates to the use of multiple water-in-oil-in-water emulsions comprising:
an external aqueous phase containing one or more hydrophilic surfactants with an ether bond such as ethylene oxide and propylene oxide copolymers, oxyethylenated fatty alcohols, or hydrophilic surfactants with an ester bond such as polyoxyethylenated sorbitan esters, the mass ratio of these surfactants with respect to the external aqueous phase being comprised between approximately 0.1 and approximately 10%.
an internal simple emulsion as defined above, comprising:
   an organic phase separating the external aqueous phase above and the internal aqueous phase below, this organic phase containing:
   one or more extractants, as defined above, the mass ratio of the extractant or extractants with respect to the organic phase being comprised between approximately 0.1 and approximately 20%,
   one or more lipophilic surfactants with an ether bond, such as the alkyl dimethicone copolyols, or with an amine bond such as long-chain condensed polyamines, or sorbitan or glycol esters, the mass ratio of the lipophilic surfactant or surfactants with respect to the organic phase being comprised between approximately 0.5 and approximately 20%,
   hydrocarbons such as liquid paraffins, perhydrosqualene or silicones or synthetic esters,
   an internal aqueous phase containing one or more de-extractants as defined above,
the external aqueous phase representing approximately 1 to approximately 80% by mass of the simple emulsion, and preferably from approximately 20% to approximately 70%.

According to an advantageous embodiment, the invention relates to the use as mentioned above, of simple or multiple emulsions, for the detoxication of acid molecules, such as acetylsalicylic acid, characterized in that the extractant is a tertiary amine, in particular trioctylamine or trilaurylamine, and in that the de-extractant is soda NaOH.

According to an advantageous embodiment, the invention relates to the use as mentioned above, of simple water-in-oil emulsions, for the detoxication of acid molecules, such as acetylsalicylic acid, comprising:
an external organic phase containing:
   liquid paraffin,
   trilaurylamine as extractant, at a rate of approximately 0.1% to approximately 3% by mass with respect to the external organic phase,
   cetyl dimethicone copolyol as lipophilic surfactant, at a rate of approximately 1 to approximately 10% by mass with respect to the external organic phase,
an internal aqueous phase containing, as de-extractant, soda, at a concentration such that the pH is greater than or equal to 13,

the mass ratio between the aqueous phase and the total emulsion being comprised between approximately 10% and approximately 70%, and preferably equal to approximately 50%.

According to an advantageous embodiment, the invention relates to the use as mentioned above, of multiple water-in-oil-in-water emulsions, for the detoxication of acid molecules, such as acetylsalicylic acid, comprising:
an external aqueous phase containing an ethylene oxide and propylene oxide copolymer as hydrophilic surfactant, at a rate of approximately 0.5 to approximately 2% by mass with respect to the total mass of the external aqueous phase,
an organic phase separating the external aqueous phase above and the internal aqueous phase below, and containing:
   liquid paraffin,
   trilaurylamine as extractant, at a rate of approximately 0.1% to approximately 3% by mass with respect to the total mass of the organic phase,
   cetyl dimethicone copolyol as lipophilic surfactant, at a rate of approximately 1 to approximately 10% by mass with respect to the total mass of the organic phase,
an internal aqueous phase containing, as de-extractant, soda, at a concentration such that the pH is greater than or equal to 13, and magnesium sulphate, at a rate of approximately 2 to approximately 6% by mass with respect to the total mass of the internal aqueous phase,

the mass ratio between the internal aqueous phase and the organic phase being comprised between approximately 25% and approximately 200%, and preferably equal to approximately 100%, and the mass ratio between the external aqueous phase and the primary simple emulsion being comprised between approximately 10% and approximately 90%, and preferably equal to approximately 25%.

According to an advantageous embodiment, the invention relates to the use as mentioned above, of simple or multiple emulsions, for the detoxication of compounds with a very slightly marked acid-basic character, such as paracetamol, characterized in that the extractant used is a long-chain alcohol, in particular octanol, and in that the de-extractant is NaOH.

According to an advantageous embodiment, the invention relates to the use as mentioned above, of simple water-in-oil emulsions comprising:
an external organic phase containing:
   liquid paraffin,
   octanol as extractant, at a rate of approximately 0.1% to approximately 20% by mass with respect to the external organic phase,
   a condensed polyamine on succinic acid substituted by a polyisobutene chain, such as ECA 4360, as lipophilic surfactant, at a rate of approximately 1 to approximately 10% by mass with respect to the external organic phase,
an internal aqueous phase containing, as de-extractant, soda at a concentration such that the pH is greater than 13,

the mass ratio between the aqueous phase and the total emulsion being comprised between approximately 10% and approximately 70%, and preferably equal to approximately 50%.

According to an advantageous embodiment, the invention relates to the use as mentioned above, of simple or multiple emulsions, for the detoxication of compounds such as zopiclone^{ND}, characterized in that the extractant used is a long-chain organothiophosphorated acid, in particular di-ethylhexyl-monothiophosphinic acid (Cyanex 302), and in that the de-extractant is hydrochloric acid.

According to an advantageous embodiment, the invention relates to the use as mentioned above, of simple water-in-oil emulsions comprising:
an external organic phase containing:
   liquid paraffin,
   Cyanex 302 as extractant, at a rate of approximately 0.1% to approximately 5% by mass with respect to the external organic phase,
   ECA 4360 as lipophilic surfactant, at a rate of approximately 1 to approximately 10% by mass with respect to the organic phase,
an internal aqueous phase containing hydrochloric acid at a concentration higher than 0.2 mol.L⁻¹, as de-extractant,

the mass ratio between the aqueous phase and the total emulsion being comprised between approximately 10% and approximately 70%, and preferably equal to approximately 50%.

The present invention relates more particularly to the use of simple or multiple emulsions as defined above, for the preparation of compositions intended for the decontamination of surfaces.

A more particular subject of the present invention is the use of simple or multiple emulsions as defined above, for the preparation of pharmaceutical compositions intended for the prevention or treatment of intoxications by oral, topical or parenteral route.

A subject of the present invention is also the use of simple or multiple emulsions as defined above, for the preparation of medical devices intended for the prevention or treatment of intoxications by oral, topical or parenteral route.

The present invention also relates to a pharmaceutical composition characterized in that it comprises a simple or multiple emulsion as defined above, if appropriate in combination with a pharmaceutically acceptable vehicle.

An advantageous pharmaceutical composition according to the invention is characterized in that it is presented in a form being able to be administered by oral route, in a single or repeated dose, in particular at a rate of approximately 10 to approximately 500 g.

An advantageous pharmaceutical composition according to the invention is characterized in that it is presented in a form being able to be administered by topical route, in particular at a rate of approximately 2 to approximately 50 mg/cm² of skin.

An advantageous pharmaceutical composition according to the invention is characterized in that it is presented in a form being able to be used for the parenteral route by extracorporeal circulation, in particular at a rate of approximately 500 to approximately 1000 g.

The present invention also relates to any multiple water-in-oil-in-water emulsion comprising in its organic phase one or more extractant compounds as defined above.

According to an advantageous embodiment of the present invention, the invention relates to any multiple emulsion as defined above, comprising in its organic phase one or more lipophilic surfactants as defined above.

An advantageous multiple emulsion of the present invention comprises in its internal aqueous phase one or more de-extractant compounds as defined above, and optional additives such as electrolytes or sugars.

The preferred electrolytes are sodium chloride or magnesium sulphate.

The preferred sugars are glucose or saccharose.

An advantageous multiple emulsion of the present invention comprises in its external aqueous phase one or more hydrophilic surfactants as defined above.

A particularly advantageous multiple emulsion of the present invention is a multiple emulsion which comprises:
an external aqueous phase containing one or more hydrophilic surfactants with an ether bond such as ethylene oxide and propylene oxide copolymers, oxyethylenated fatty alcohols, or hydrophilic surfactants with an ester bond such as polyoxyethylenated sorbitan esters, the mass ratio of these surfactants with respect to the external aqueous phase being comprised between approximately 0.1 and approximately 10%.
an internal simple emulsion as defined above, comprising:
   an organic phase separating the external aqueous phase above and the internal aqueous phase below, this organic phase containing:
   one or more extractants, as defined above, the mass ratio of the extractant or extractants with respect to the organic phase being comprised between approximately 0.1 and approximately 20%,
   one or more lipophilic surfactants with an ether bond, such as the alkyl dimethicone copolyols, or with an amine bond such as long-chain condensed polyamines, or sorbitan or glycol esters, the mass ratio of the lipophilic surfactant or surfactants with respect to the organic phase being comprised between approximately 0.5 and approximately 20%,
   hydrocarbons such as liquid paraffins, perhydrosqualene or silicones or synthetic esters,
   an internal aqueous phase containing one or more de-extractants as defined above,

the external aqueous phase representing approximately 1 to approximately 80% by mass of the simple emulsion, and preferably from approximately 10% to approximately 70%.

The present invention also relates to a multiple emulsion as defined above, for the detoxication of acid molecules, such as acetylsalicylic acid, comprising:
an external aqueous phase containing an ethylene oxide and propylene oxide copolymer as hydrophilic surfactant, at a rate of approximately 0.5 to approximately 2% by mass with respect to the total mass of the external aqueous phase,
an organic phase separating the external aqueous phase above and the internal aqueous phase below, and containing:
   liquid paraffin,
   trilaurylamine as extractant, at a rate of approximately 0.1% to approximately 3% by mass with respect to the total mass of the organic phase,
   cetyl dimethicone copolyol as lipophilic surfactant, at a rate of approximately 1 to approximately 10% by mass with respect to the total mass of the organic phase,
an internal aqueous phase containing soda as de-extractant, at a concentration such that the pH is greater than or equal to 13, and magnesium sulphate, at a rate of approximately 2 to approximately 6% by mass with respect to the total mass of the internal aqueous phase,

the mass ratio between the internal aqueous phase and the organic phase being comprised between approximately 25% and approximately 200%, and preferably equal to approximately 100%, and the mass ratio between the external aqueous phase and the primary simple emulsion being comprised between approximately 10% and approximately 90%, and preferably equal to approximately 25%.

### EXPERIMENTAL PART

The principle of the extraction of toxic molecules by an emulsion system (water-in-oil emulsion or water-in-oil-in water emulsion) consists of:

1) introducing into the organic phase a lipophilic molecule which will play the role of an extractant by reacting chemically with the toxic molecule at the interface with the external aqueous phase, and by forming with the latter a liposoluble compound which passes through the organic membrane, under the effect of its concentration gradient.

2) incorporating into the internal aqueous phase a molecule which will react chemically at the internal interface with the lipophilic complex formed previously, which, on the one hand, makes it possible to regenerate the extractant and, on the other hand, to trap the toxic molecule in the internal phase.

Definition and Preparation of the Systems

Two systems are envisaged: a simple water-in-oil emulsion (system I) and a multiple water-in-oil-in-water emulsion (system II).

The preparation of system I is carried out in two phases:

1) during the first phase, a simple water-in-oil emulsion is prepared. This simple emulsion is stabilized by a lipophilic surfactant with a low HLB (Hydrophile/Lipophile Balance) and contains fine droplets of internal aqueous phase with a diameter of 0.5 to 1 μm. It is presented as a relatively viscous milk.

2) during the second phase, the simple emulsion obtained previously is in turn dispersed in the solution containing the toxic compound. This second stage requires the intervention of moderate stirring, provided by a magnetic stirrer, in the in vitro study.

The preparation of system II is also carried out in two phases:

1) a first phase identical to that implemented for system I,

2) a second phase during which the simple emulsion (called primary) is dispersed in an aqueous solution containing a hydrophilic surfactant. The multiple emulsion thus obtained is then dispersed in the solution containing the toxic compound.

Principle of the Extraction

During the contact between the emulsion system and the solution, the toxic compound will pass through the organic solution to be collected and trapped in the droplets of internal aqueous phase.

The originality and the effectiveness of the process of the present invention rests on two particularly important features: the presence in the organic solution of a molecule (transporter or extractant) capable of assisting with the transport of the toxic compound in this phase and the presence in the internal aqueous phase of a trapping agent (de-extractant) which destroys the transporter-toxic substance complex and reacts with the latter in order to convert it into in a very lipophobic species. The transporter thus regenerated diffuses in order to react with a new molecule of toxic compound.

### Example of Aspirin (Acetylsalicylic Acid)

I—Theoretical Note:

Acetylsalicylic acid is called HA in an effort to simplify matters. In this case, the transporter is a long-chain tertiary amine (R₃N), a molecule with a weak basic character, which is very slightly soluble in water.

At the first interface between the external aqueous phase and the organic phase, the following chemical reaction is produced:
HA_{aq}+R₃N_{org}⇄R₃NHA_{org}

The R₃NHA molecule is much more lipophilic than HA and therefore facilitates its solubilization in the organic phase.

The trapping agent introduced into the internal aqueous phase is soda. At the second interface between the organic phase and the internal aqueous phase, the following chemical reaction is produced:
R₃NHA_{org}+OH⁻_{aq}⇄R₃N_{org}+A⁻_{aq}+H₂O

The acetylsalicylic acid is thus converted into acetylsalicylate ion, totally insoluble in the organic phase. The aspirin is thus trapped in the internal phase and R₃N, regenerated, starts a new cycle.

Such a chemical system is also valuable for other toxic molecules with an acid character. The effectiveness of such systems has been demonstrated for organic acids such as lactic acid, ascorbic acid or citric acid, and for strong or weak mineral acids, such as hydrochloric acid, nitric acid, sulphuric acid, hydrofluoric acid or hydrocyanic acid. Primary, secondary and tertiary amines can be used as transporter, providing that they have one or more long carbon chains (in order to minimize their solubility in water). However, the tertiary amines are the most effective as they are the most basic.

The internal aqueous solution is a basic solution, constituted by a strong base such as soda or potash or by a weak base such as sodium carbonate or potassium carbonate. However, the strong bases are the most effective.

II—Evaluation of the Properties of Extraction of Acetylsalicylic Acid by Simple Emulsions (System I) According to the Nature and Concentration of the Constituents:

Different components of the emulsion have been studied:
Apolar organic solvent: dodecane or hydrogenated polyisobutene (parleam)
organic extractant: trioctylamine (C₈H₁₇)₃N or trilaurylamine (C₁₂H₂₅)₃N
surfactant: ECA 4630 or Abil EM 90
internal phase of the emulsion: soda

A) Preparation of the Emulsions:

The two phases are emulsified for 3 minutes using an Ultraturrax turning at 13,500 rpm. The average diameter of the drops of the emulsion thus obtained is of the order of 0.5 to 1 μm.

B) Extraction In Vitro:

50 mL of emulsion is brought into contact, in a beaker, under mechanical stirring, with 100 mL of an aqueous solution containing 0.02 or 0.01 mol.L⁻¹ of aspirin and the residual concentration of the aspirin in the external aqueous phase is monitored over time. The extraction percentage, which is a parameter allowing comparison of the effectiveness of the different emulsions prepared, is then deduced from this.

1) Influence of Soda

In order to determine the influence of soda, emulsions containing the following components are prepared:
Dodecane 46.5%
TLA 2.5%
Abil 1%
NaOH at different concentrations: 50% (hereafter, all percentages represent mass ratios)

After contact for 3 minutes, the following results are obtained:

NaOH mol · L⁻¹

| | 0 | 0.1 | 0.3 | 1 |
|---|---|---|---|---|
| % extraction | 40 | 97 | 93 | 94 |

Thus the importance of the presence of soda in the internal phase is observed. Above 0.1 mol.L⁻¹, the concentration no longer has any effect, to the extent that the OH⁻/aspirin stoichiometric quantity is respected.

2) Influence of the Concentration of Extractant

In order to determiner the role of the concentration of extractant, the 3 following formulations are compared; they all contain 2% of Abil and 50% of NaOH 0.1 mol.L⁻¹ and:

| | Emulsion A | Emulsion B | Emulsion C |
|---|---|---|---|
| Dodecane % | 48 | 47.5 | 47 |
| (solvent) | | | |
| TOA % | 0 | 0.5 | 1 |
| (extractant) | | | |

The extraction percentage as a function of time for the 3 emulsions is given below:

| Time | % extraction | % extraction | % extraction |
|---|---|---|---|
| (minutes) | Emulsion A | Emulsion B | Emulsion C |
| 0 | 0 | 0 | 0 |
| 1 | 27 | 70 | 72 |
| 2 | 47 | 90 | 92 |
| 4 | 73 | 98 | 99 |
| 6 | 86 | >99 | >99 |
| 8 | 93 | >99 | >99 |

It is therefore noted that the performances are very distinctly higher in the presence of the extractant. In fact, the extraction is total in less than 3 minutes in the presence of TOA, whereas it is necessary to wait 8 minutes in order to have more than 90% extraction without TOA.

In the case of TLA, the following emulsions are prepared:

| | Emulsion A | Emulsion B | Emulsion C |
|---|---|---|---|
| Dodecane % | 44 | 43.5 | 43 |
| TLA % | 0 | 0.5 | 1 |
| Abil % | 6 | 6 | 6 |
| NaOH 0.1 mol · L⁻¹ % | 50 | 50 | 50 |

It is noted that the results obtained are similar:

| Time | % extraction | % extraction | % extraction |
|---|---|---|---|
| (minutes) | Emulsion A | Emulsion B | Emulsion C |
| 0 | 0 | 0 | 0 |
| 2 | 45 | 55 | 55 |
| 4 | 70 | 84 | 85 |
| 6 | 80 | 91 | 92 |
| 8 | 90 | 96 | 96 |
| 10 | 93 | >99 | >99 |

3) Influence of the Concentration of Surfactant

In order to determine the influence of the concentration of surfactant, the following emulsions, containing trilaurylamine (TLA) are prepared:

Emulsion

| | A | B | C | D |
|---|---|---|---|---|
| Dodecane % | 46.6 | 44.6 | 41.8 | 38 |
| TLA % | 2.4 | 2.4 | 2.2 | 2 |
| Abil % | 1 | 3 | 6 | 10 |
| NaOH 0.1 mol · L⁻¹ % | 50 | 50 | 50 | 50 |

The following results are obtained after three minutes:

Emulsion

| | A | B | C | D |
|---|---|---|---|---|
| % extraction | 55 | 88 | 80 | 76 |

The optimum content of the surfactant is therefore of the order of 3 to 5% of Abil.

Similarly, the following emulsions, containing trioctylamine (TOA) are prepared:

| Emulsion | A | B | C | D | E | F |
|---|---|---|---|---|---|---|
| Dodecane % | 47 | 44.1 | 42.1 | 39.2 | 34.3 | 29.4 |
| TOA % | 1 | 0.9 | 0.9 | 0.8 | 0.7 | 0.6 |
| Abil % | 2 | 5 | 7 | 10 | 15 | 20 |
| NaOH 0.1 mol · L⁻¹ % | 50 | 50 | 50 | 50 | 50 | 50 |

The effectiveness of the extraction is compared after 2 minutes, and the following results are obtained:

| Emulsion | A | B | C | D | E | F |
|---|---|---|---|---|---|---|
| % extraction | 82 | 92 | 98 | 93 | 93 | 92 |

The optimum is less distinct, but exists, around 7% of Abil.

4) Influence of the Quantity of Emulsion

For 100 mL of aspirin solution containing 0.01 mol.L⁻¹, different volumes of emulsion were used: 15, 33 and 50 mL, which corresponds to volume ratios of 6.5; 3 and 2.

The composition of the prepared emulsion is the following:
Dodecane: 47.1%
TOA: 0.9%
ABIL: 2%
NaOH 0.1 mol.L⁻¹: 50%

The extraction percentages obtained are the following:

| | | | | % extraction |
|---|---|---|---|---|
| Time | % extraction | % extraction | % extraction | ratio 6.5 soda |
| (minutes) | ratio 6.5 | ratio 3 | ratio 2 | 0.16 mol · L⁻¹ |
| 0 | 0 | 0 | 0 | 0 |
| 1 | 44 | 65 | 73 | 44 |
| 2 | 55 | 92 | 93 | 75 |
| 4 | 67 | 98 | 99 | 90 |
| 6 | 70 | >99 | >99 | 95 |
| 8 | 70 | >99 | >99 | 98 |

In the case of the ratio 6.5, the excess of soda is relatively small which can explain the slowing down of the transfer. Thus, a test was carried out with the same ratio, but with soda at 0.16 mol.L⁻¹. The extraction becomes almost total in 8 minutes, but it is slower than with lower volume ratios (interface area smaller during the extraction when the volume ratio increases).

The table below shows examples of effective emulsions, with a volume ratio equal to 2:

| | | | | |
|---|---|---|---|---|
| Solvent | Dodecane | Dodecane 47% | Dodecane | Parleam 44.7% |
| | 42.1% | | 44.7% | |
| Extractant | TOA 0.9% | TOA 1% | TLA 2.3% | TLA 2.3% |
| Surfactant | Abil 7% | ECA 2% | Abil 3% | Abil 3% |
| NaOH 0.1 | 50% | 50% | 50% | 50% |
| mol · L⁻¹ | | | | |
| Total | 3 minutes | 3 minutes | 5 minutes | 4 minutes |
| extraction | | | | |
| time | | | | |

All these emulsions show a failure rate of less than 1% after 15 minutes of stirring, attesting to their stability during their use.

III—Generalization and Optimization in the Case of Multiple Emulsions (System II):

The standard formula adopted contains the following components:

Primary Emulsion:
Apolar organic solvent: liquid paraffin (Primol 352)
Organic extractant: Trilaurylamine (C₁₂H₂₅)₃N
Primol 352+TLA: 30%
Surfactant: Cetyl dimethicone copolyol (Abil EM 90): 6%
Trapping agent: Soda
Electrolyte: Magnesium sulphate (MgSO₄)

Multiple Emulsion:
hydrophilic surfactant: Arlatone F127G (ethylene oxide and propylene oxide copolymer): 1%
primary emulsion: 80% in the multiple emulsion

These constituents, as well as the indicated values of the concentrations, were adopted taking into consideration the studies carried out with the simple emulsions, from the perspective of the capture of acetylsalicylic acid, as well as the earlier results obtained on the stability of the multiple emulsions. Certain modifications with respect to the formulae of simple emulsions have however been made, for the following reasons:

Concerning the choice of the oil, comparative studies have made it possible to show that the effectiveness of extraction through the lipid membrane was practically unchanged for most of the solvents. However, a liquid paraffin was preferred because of its well-known harmlessness by oral route and its non-digestibility. Similarly, the choice of the organic extractant was trilaurylamine because of its lower toxicity by oral route.

An electrolyte (magnesium sulphate) on the other hand was introduced into the internal aqueous phase, in order to stabilize the interface between the aqueous microglobules and the organic phase. Earlier studies carried out on multiple emulsions in fact showed the stabilizing role of certain electrolytes. Finally, since the system contains two interfaces, it was necessary to introduce a hydrophilic surfactant into the external aqueous phase of the multiple emulsion. The concentration of this surfactant was fixed at 1%, because of earlier studies which established that this value ensured maximum stability.

A) Preparation of the Multiple Emulsions:

The primary emulsion is prepared by heating the internal aqueous phase and the organic phase to 70-80° C. using a water bath. The aqueous phase is incorporated into the oily phase under vigorous stirring at 3000 rpm using a centripetal-type Rayneri turbine mixer for 30 minutes. Then, after cooling down to ambient temperature, the primary emulsion is introduced slowly into the external aqueous phase. This second emulsification is ensured by the same turbine mixer under stirring at 500 rpm. The duration of stirring is a function of the formulation and can vary from 5 to 45 minutes.

B) Study of the Influence of the Concentration of the Extractant, the Trapping Agent and the Electrolyte on the In Vitro Extraction of Acetylsalicylic Acid:

The same procedure is used as for system 1.10 mL of emulsion is brought into contact in a beaker, under mechanical stirring, with 50 mL of an aqueous solution containing 0.02 mol.L⁻¹ of aspirin; the residual concentration of the aspirin in the external aqueous phase is monitored over time. The extraction percentage, which is a parameter allowing comparison of the effectiveness of the different emulsions, is then deduced from this.

In order to obtain a comparative reference, the extraction levels at different times were first measured using a “white” emulsion, thus named because it contains none of the constituents the action of which is supposed to be indispensable for good extraction, namely the extracting and trapping agents. The formula of this white emulsion, as well as the extraction levels are given in the tables below (hereafter, the percentages are calculated with respect to the primary emulsion):

| | % |
|---|---|
| Primol | 30 |
| MgSO4 | 0.7 |
| Distilled water | Qs 100 |
| Time (minutes) | % extraction |
| 1 | 21 |
| 5 | 40 |
| 10 | 42 |
| 15 | 43 |

1) Influence of the Concentration of Trapping Agent (NaOH)

3 emulsions are prepared, differing only in their NaOH concentration:

| % | Emulsion A | Emulsion B | Emulsion C |
|---|---|---|---|
| Primol + TLA (0.3%) | 30 | 30 | 30 |
| MgSO₄ | 3 | 3 | 3 |
| NaOH qs 100 | 0.1 mol · L⁻¹ | 0.2 mol · L⁻¹ | 0.3 mol · L⁻¹ |

The following results are obtained:

| Time | % extraction | % extraction | % extraction |
|---|---|---|---|
| (minutes) | Emulsion A | Emulsion B | Emulsion C |
| 1 | 42 | 50 | 58 |
| 5 | 50 | 50 | 61 |
| 10 | 50 | 50 | 58 |
| 15 | 50 | 50 | 60 |

An increase in the effectiveness of extraction is therefore observed with the concentration of trapping agent, even above the concentration 0.1 mol.L⁻¹. This may appear surprising since the results obtained on the simple emulsions had made it possible to show that the maximum effectiveness level was reached starting from 0.1 mol.L⁻¹, a concentration for which the equality of the OH⁻/aspirin stoichiometric concentration was obtained.

2) Influence of the Concentration of Electrolyte (MgSO₄)

The following 3 formulae were prepared, which differ only in their MgSO₄ concentration:

| % | Emulsion A | Emulsion B | Emulsion C |
|---|---|---|---|
| Primol + TLA (0.3%) | 30 | 30 | 30 |
| MgSO₄ | 1 | 2 | 3 |
| NaOH 0.2 mol · L⁻¹ | QS 100 | QS 100 | QS 100 |

The extraction percentage as a function of time for the 3 emulsions is given below:

| Time | % extraction | % extraction | % extraction |
|---|---|---|---|
| (minutes) | Emulsion A | Emulsion B | Emulsion C |
| 1 | 55 | 65 | 47 |
| 5 | 60 | 65 | 47 |
| 10 | 60 | 65 | 47 |
| 15 | 60 | 65 | 47 |

It is observed that the extraction is maximal for an electrolyte concentration of 2%. However, it is noted that the extraction level still remains relatively low, although it is significantly higher than that of the white emulsion (45%).)

3) Influence of the Concentration of Extractant (TLA)

4 formulae are compared, which differ only in their TLA concentration:

| | Emulsion | Emulsion | Emulsion | Emulsion |
|---|---|---|---|---|
| % | A | B | C | D |
| TLA | 0.3 | 1 | 1.5 | 2.4 |
| Primol + TLA | 30 | 30 | 30 | 30 |
| MgSO₄ | 2 | 2 | 2 | 2 |
| NaOH 0.2 mol · L⁻¹ | Qs 100 | Qs 100 | Qs 100 | Qs 100 |

The extraction percentages as a function of time for the 4 emulsions are given below:

| | % extraction | % extraction | % extraction | % extraction |
|---|---|---|---|---|
| Time (minutes) | Emulsion A | Emulsion B | Emulsion C | Emulsion D |
| 1 | 65 | 92 | 95 | 82 |
| 5 | 65 | 88 | 92 | 80 |
| 10 | 65 | 86 | 90 | 78 |
| 15 | 65 | 84 | 90 | 76 |

As with system I, it is observed that the performances are very distinctly higher when the extractant has a suitable concentration, not so much with respect to the extraction level as to its effectiveness. Whereas the extraction level does not exceed 65% when the TLA concentration is 0.3%, it reaches values of the order of 90% when it is comprised between 1 and 1.5%. It clearly appears that the optimum concentration is approximately 1.5%. It should be noted that above 1.5% of TLA, the stability of the multiple emulsion reduces, which leads to a reduction in effectiveness.

Example of effective multiple emulsion (volume ratio 2):

| | % |
|---|---|
| TLA | 1.5 |
| Primol + TLA | 30 |
| MgSO₄ | 2 |
| NaOH 0.2 mol · L⁻¹ | Qs 100 |

Since oral administration is envisaged, it is evidently essential to ensure that the emulsion systems considered will preserve their stability in gastric and intestinal medium. In vitro studies relating to multiple white emulsions (in the absence of an extractant and trapping agent pair) in aqueous phases having the same composition as the gastric and intestinal media, have already demonstrated a remarkable stability vis-à-vis the digestive enzymes.

### Example of Paracetamol

For toxic molecules with a very weak acid-basic character (alcohol, glycol; paracetamol for example), a solvating transporter and internal aqueous phase capable of reacting with the toxic substance in order to trap it in a lipophobic form (oxidizing agent, strong base etc.) are envisaged.

Experiments have been carried out within the framework of the detoxication of paracetamol.

The following emulsion was prepared:

Organic Phase
octanol: 20%
ECA 4360 (surfactant): 3%
dodecane qs

Internal Aqueous Phase
NaOH 0.5 mol.L⁻¹

An extraction effectiveness of 80% is obtained in less than 5 minutes.

### Example Of Zopiclone

Symmetrical systems are envisaged in order to extract toxic substances with a basic character using an organic acid molecule (organophosphorated acid, carboxylic acid) as transporter which is very slightly soluble in water; the internal aqueous solution is an acid solution.

The reactions involved are the following; for example for ammonia:

1^{st} interface: NH_{3aq}+H_{org}⇄NH₄R_{org}

2^{nd} interface: NH₄R_{org}+H⁺_{aq}⇄HR_{org}+NH₄⁺_{aq}

Experiments were carried out within the framework of detoxication of zopiclone, which is a molecule with a basic character.

The following emulsion was prepared:

Organic Phase
Cyanex 302: 2%
ECA 4360 (surfactant): 10%
dodecane qs

Internal Aqueous Phase
HCl 1 mol.L⁻¹

An extraction effectiveness of 90% is obtained in 5 minutes.

1. Use of simple or multiple emulsions comprising in their organic phase one or more extractant compounds capable, when said emulsions are brought into contact with a medium, either biological such as gastric liquid, skin or blood, or artificial such as metal or plastic surfaces, of extracting from said medium specific toxic molecules capable of binding to said extractant or extractants, for the preparation of pharmaceutical compositions intended for the prevention or treatment of intoxications by oral, topical or parenteral route, or for the detoxication of surfaces by simple application to the abovementioned surfaces.

2. Use according to claim 1, of simple water-in-oil, or multiple water-in-oil-in-water emulsions, the internal aqueous phase of which comprises one or more de-extractant compounds, trapping the toxic molecules extracted from the medium.

3. Use according to claim 1, characterized in that the extractant is chosen from:
amine derivatives, such as the primary, secondary or tertiary amines or quaternary ammonium salts, comprising one or more carbon chains each comprising approximately 1 to 18 carbon atoms, in particular trioctylamine or trilaurylamine, when the toxic molecule to be eliminated has an acid or anionic character,
organic acids, such as organophosphorated acids, thiophosphorated acids, carboxylic acids, comprising one or more carbon chains each comprising approximately 1 to 18 carbon atoms, when the toxic molecule to be eliminated has a basic or cationic character,
solvating molecules, such as alcohols, organophosphates, phosphine oxides, organosulphides or sulphoxides, comprising one or more carbon chains each comprising approximately 1 to 18 carbon atoms, when the toxic molecule to be eliminated has a neutral character.

4. Use according to claim 1, characterized in that the de-extractant is chosen from:
bases such as NaOH, KOH, Na₂CO₃, when the toxic molecule to be eliminated has an acid or anionic character,
ionic salts such as NaCl, NH₄Cl or NaNO₃, when the toxic molecule to be eliminated has an anionic character,
acids such as hydrochloric acid or lactic acid, when the toxic molecule to be eliminated has a basic or cationic character,
compounds which are oxide-reducing or chelating in character, such as chromium (VI) salts, thiourea, ethylene diamine tetracetic acid, chlorinated or fluorinated derivatives, ascorbic acid, when the toxic molecule to be eliminated has a neutral character.

5. Use according to claim 1, of simple water-in-oil emulsions comprising:
an external organic phase containing:
one or more extractants, as defined in claim 3, the mass ratio of the extractant or extractants with respect to the organic phase being comprised between approximately 0.1 and approximately 20%,
one or more lipophilic surfactants with an ether bond, such as the alkyl dimethicone copolyols, or with an amine bond such as long-chain condensed polyamines, or sorbitan or glycol esters, the mass ratio of the lipophilic surfactant or surfactants with respect to the organic phase being comprised between approximately 0.5 and approximately 20%,
hydrocarbons (qs) such as liquid paraffins, perhydrosqualene or silicones or synthetic esters,
an internal aqueous phase containing one or more de-extractants as defined in claim 4, and optionally an additive such as an electrolyte or a sugar, the mass ratio of the internal aqueous phase with respect to the emulsion being comprised between approximately 1 and approximately 80%, preferably between approximately 20% and approximately 70%.

6. Use according to claim 1, of multiple water-in-oil-in-water emulsions comprising:
an external aqueous phase containing one or more hydrophilic surfactants with an ether bond such as ethylene oxide and propylene oxide copolymers, oxyethylenated fatty alcohols, or hydrophilic surfactants with an ester bond such as polyoxyethylenated sorbitan esters, the mass ratio of these surfactants with respect to the external aqueous phase being comprised between approximately 0.1 and approximately 10%,
an internal simple emulsion as defined in claim 5, comprising:
an organic phase separating the external aqueous phase above and the internal aqueous phase below, this organic phase containing:
one or more extractants, as defined in claim 3, the mass ratio of the extractant or extractants with respect to the organic phase being comprised between approximately 0.1 and approximately 20%,
one or more lipophilic surfactants with an ether bond, such as the alkyl dimethicone copolyols, or with an amine bond such as long-chain condensed polyamines, or sorbitan or glycol esters, the mass ratio of the lipophilic surfactant or surfactants with respect to the organic phase being comprised between approximately 0.5 and approximately 20%,
hydrocarbons such as liquid paraffins, perhydrosqualene or silicones or synthetic esters,
an internal aqueous phase containing one or more de-extractants,
the external aqueous phase representing approximately 1 to approximately 80% by mass of the simple emulsion, and preferably from approximately 20% to approximately 70%.

7. Use according to claim 1, of simple or multiple emulsions, for the detoxication of acid molecules, such as acetylsalicylic acid, characterized in that the extractant is a tertiary amine, in particular trioctylamine or trilaurylamine, and in that the de-extractant is soda NaOH.

8. Use according to claim 7, of simple water-in-oil emulsions, for the detoxication of acid molecules, such as acetylsalicylic acid, comprising:
an external organic phase containing:
liquid paraffin,
trilaurylamine as extractant, at a rate of approximately 0.1% to approximately 3% by mass with respect to the external organic phase,
cetyl dimethicone copolyol as lipophilic surfactant, at a rate of approximately 1 to approximately 10% by mass with respect to the external organic phase,
an internal aqueous phase containing, as de-extractant, soda, at a concentration such that the pH is greater than or equal to 13,
the mass ratio between the aqueous phase and the total emulsion being comprised between approximately 10% and approximately 70%, and preferably equal to approximately 50%.

9. Use according to claim 7, of multiple water-in-oil-in-water emulsions, for the detoxication of acid molecules, such as acetylsalicylic acid, comprising:
an external aqueous phase containing an ethylene oxide and propylene oxide copolymer as hydrophilic surfactant, at a rate of approximately 0.5 to approximately 2% by mass with respect to the total mass of the external aqueous phase,
an organic phase separating the external aqueous phase above and the internal aqueous phase below, and containing:
liquid paraffin,
trilaurylamine as extractant, at a rate of approximately 0.1% to approximately 3% by mass with respect to the total mass of the organic phase,
cetyl dimethicone copolyol as lipophilic surfactant, at a rate of approximately 1 to approximately 10% by mass with respect to the total mass of the organic phase,
an internal aqueous phase containing, as de-extractant, soda, at a concentration such that the pH is greater than or equal to 13, and magnesium sulphate, at a rate of approximately 2 to approximately 6% by mass with respect to the total mass of the internal aqueous phase,
the mass ratio between the internal aqueous phase and the organic phase being comprised between approximately 25% and approximately 200%, and preferably equal to approximately 100%, and the mass ratio between the external aqueous phase and the primary simple emulsion being comprised between approximately 10% and approximately 90%, and preferably equal to approximately 25%.

10. Use according to claim 1, of simple or multiple emulsions, for the detoxication of compounds with a very slightly marked acid-basic character, such as paracetamol, characterized in that the extractant used is a long-chain alcohol, in particular octanol, and in that the de-extractant is NaOH.

11. Use according to claim 10, of simple water-in-oil emulsions comprising:
an external organic phase containing:
liquid paraffin,
octanol as extractant, at a rate of approximately 0.1% to approximately 20% by mass with respect to the external organic phase,
a condensed polyamine on succinic acid substituted by a polyisobutene chain, such as ECA 4360, as lipophilic surfactant, at a rate of approximately 1 to approximately 10% by mass with respect to the external organic phase,
an internal aqueous phase containing, as de-extractant, soda at a concentration such that the pH is greater than 13,
the mass ratio between the aqueous phase and the total emulsion being comprised between approximately 10% and approximately 70%, and preferably equal to approximately 50%.

12. Use according to claim 1, of simple or multiple emulsions, for the detoxication of compounds such as zopiclone, characterized in that the extractant used is a long-chain organothiophosphorated acid, in particular di-ethylhexyl-monothiophosphinic acid (Cyanex 302), and in that the de-extractant is hydrochloric acid.

13. Use according to claim 12, of simple water-in-oil emulsions comprising:
an external organic phase containing:
liquid paraffin,
Cyanex 302 as extractant, at a rate of approximately 0.1% to approximately 5% by mass with respect to the external organic phase,
ECA 4360 as lipophilic surfactant, at a rate of approximately 1 to approximately 10% by mass with respect to the organic phase,
an internal aqueous phase containing hydrochloric acid at a concentration higher than 0.2 mol.L⁻¹, as de-extractant,
the mass ratio between the aqueous phase and the total emulsion being comprised between approximately 10% and approximately 70%, and preferably equal to approximately 50%.

14. Pharmaceutical composition characterized in that it comprises a simple or multiple emulsion as defined in claim 1, if appropriate in combination with a pharmaceutically acceptable vehicle.

15. Pharmaceutical composition according to claim 14, characterized in that it is presented in a form being able to be administered by oral route, in a single or repeated dose, in particular of approximately 10 to approximately 500 g.

16. Pharmaceutical composition according to claim 14, characterized in that it is presented in a form being able to be administered by topical route, in particular at a dose of approximately 2 to approximately 50 mg/cm² of skin.

17. Pharmaceutical composition according to claim 14, characterized in that it is presented in a form being able to be administered by parenteral route by extracorporeal circulation, in particular at a dose of approximately 500 to approximately 1000 g.

18. Multiple water-in-oil-in-water emulsion comprising in its organic phase one or more extractant compounds as defined in claim 1.

19. Multiple emulsion according to claim 18, comprising in its organic phase one or more lipophilic surfactants.

20. Multiple emulsion according to claim 18, comprising in its internal aqueous phase one or more de-extractant compounds.

21. Multiple emulsion according to claim 18, comprising in its external aqueous phase one or more hydrophilic surfactants.

22. Multiple water-in-oil-in-water emulsion according to claim 18, comprising:
an external aqueous phase containing one or more hydrophilic surfactants with an ether bond such as ethylene oxide and propylene oxide copolymers, oxyethylenated fatty alcohols, or hydrophilic surfactants with an ester bond such as polyoxyethylenated sorbitan esters, the mass ratio of these surfactants with respect to the external aqueous phase being comprised between approximately 0.1 and approximately 10%.
an internal simple emulsion comprising:
an organic phase separating the external aqueous phase above and the internal aqueous phase below, this organic phase containing:
one or more extractants, as defined in claim 3, the mass ratio of the extractant or extractants with respect to the organic phase being comprised between approximately 0.1 and approximately 20%,
one or more lipophilic surfactants with an ether bond, such as the alkyl dimethicone copolyols, or with an amine bond such as long-chain condensed polyamines, or sorbitan or glycol esters, the mass ratio of the lipophilic surfactant or surfactants with respect to the organic phase being comprised between approximately 0.5 and approximately 20%,
hydrocarbons such as liquid paraffins, perhydrosqualene or silicones or synthetic esters,
an internal aqueous phase containing one or more de-extractants,
the external aqueous phase representing approximately 1 to approximately 80% by mass of the simple emulsion, and preferably from approximately 10% to approximately 70%.

23. Multiple emulsion according to claim 18, for the detoxication of acid molecules, such as acetylsalicylic acid, comprising:
an external aqueous phase containing an ethylene oxide and propylene oxide copolymer as hydrophilic surfactant, at a rate of approximately 0.5 to approximately 2% by mass with respect to the total mass of the external aqueous phase,
an organic phase separating the external aqueous phase above and the internal aqueous phase below, and containing:
liquid paraffin,
trilaurylamine as extractant, at a rate of approximately 0.1% to approximately 3% by mass with respect to the total mass of the organic phase,
cetyl dimethicone copolyol as lipophilic surfactant, at a rate of approximately 1 to approximately 10% by mass with respect to the total mass of the organic phase,
an internal aqueous phase containing as de-extractant soda, at a concentration such that the pH is greater than or equal to 13, and magnesium sulphate, at a rate of approximately 2 to approximately 6% by mass with respect to the total mass of the internal aqueous phase,
the mass ratio between the internal aqueous phase and the organic phase being comprised between approximately 25% and approximately 200%, and preferably equal to approximately 100%, and the mass ratio between the external aqueous phase and the primary simple emulsion being comprised between approximately 10% and approximately 90%, and preferably equal to approximately 25%.
